(19) United States
(12) Patent Application Publication
(10) Pub. No.: US 2010/0076454 A1
(43) Pub. Date: Mar. 25, 2010

(54) POSITIONING DEVICE FOR POSITIONING AN OBJECT ON A SURFACE

(75) Inventor: Dennis Erwin BOS, EINDHOVEN (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/529,368

(22) PCT Filed: Mar. 3, 2008
(86) PCT No.: PCT/IB2008/050761

§ 371 Date: Sep. 1, 2009

(30) Foreign Application Priority Data
Mar. 7, 2007 (EP) 07103678.4

(51) Int. Cl. A61B 19/00

(52) U.S. Cl. 606/130

(57) ABSTRACT

The present invention relates to a positioning device for positioning an object (6) on a surface (7). The positioning device comprises a provision unit for providing the position of a first position on the surface (7) and a moving unit (5) for moving the object (6) on the surface (7) from the first location to a second location along a given path, wherein the object (6) is continuously in contact with the surface (7) during the movement of the object (6) on the surface (7) from the first location to the second location.

### FIELD OF THE INVENTION

The invention relates to a positioning device, a positioning method and a computer program for positioning an object on a surface. The invention relates further to a catheter apparatus comprising the positioning device and to a corresponding catheter method and computer program.

### BACKGROUND OF THE INVENTION

A catheter system for ablation within a heart of a patient comprises a catheter tip including an ablation electrode for ablating tissue of the heart. A user tries to position the catheter tip on a desired location on a moving inner wall, i.e. on a moving inner surface, of the heart by hand. After positioning of the catheter tip, a X-ray fluoroscopy system determines the actual position of the catheter tip on the moving inner wall of the heart, and, since the determined position, i.e. the actual position, of the catheter tip does generally not coincide with the desired location on the inner moving wall, the catheter tip has to be positioned again, and also the determination of the actual position using the X-ray fluoroscopy system has to be repeated. This procedure is continued, until the catheter tip has been positioned correctly on the moving inner wall. After the correct positioning, tissue is ablated at the position of the catheter tip. Subsequent points are ablated by repeating this procedure, whereas typically the points together need to form a line segment in order to result in effective treatment. This positioning of the catheter tip on a moving inner surface is inaccurate and requires several determinations of the actual catheter tip position using an X-ray fluoroscopy system, which results in a large dose applied to a patient. Even if a user tries to position the catheter tip on a desired location on a surface, which is not moving, the positioning of the catheter tip is inaccurate and requires several determinations of the actual catheter tip position, because the positioning is disturbed by the flow of blood.

### SUMMARY OF THE INVENTION

It is an object of the present invention to provide a positioning device, a positioning method and a computer program for positioning an object on a surface, wherein the positioning is improved, in particular wherein the positioning is more accurate.

In an aspect of the present invention a positioning device for positioning an object on a surface is provided, wherein the positioning device comprises:

a provision unit for providing the position of a first location on the surface,

a moving unit for moving the object on the surface from the first location to a second location along a given path, wherein the object is continuously in contact with the surface during the movement of the object on the surface from the first location to the second location.

The invention is based on the idea that, since the object is continuously in contact with the surface and since, thus, the spatial relationship between the object and the surface does not get lost during the movement of the object from the first location to the second location, the positioning of the object on the surface is improved, in particular the accuracy of the positioning is improved. Thus, for example, even if the surface is a moving surface or if the object has to withstand the flow of fluids like blood or other influences acting on the object and/or the surface, the positioning of the object on the surface is improved due to the continuous contact between the object and the surface.

The contact between the object and the surface does not have to be direct. The contact can also be indirect, for example, the object can be attached to an element, which contacts the surface, wherein the attachment and the element is designed such that the spatial relationship between the object and the element and, therefore, the spatial relationship between the object and the surface is known, in particular this element and the attachment are rigid.

The object is, for example, a catheter tip, and the surface is, for example, a surface of an inner wall of a heart of a patient.

In a preferred embodiment, the positioning device further comprises a position determination unit for determining the position of the second location on the surface from the provided position of the first location and from the given path.

Since the position of the first location is provided by the provision unit, which is preferentially an X-ray fluoroscopy system, and since the path from the first location to the second location is given and, therefore, known, the position of the second location can easily be determined, for example, by vectorially adding the provided position of the first location and the given path.

In a further embodiment, the moving unit is adapted for moving the object from the first location to a second location of a plurality of second locations along a first given path and for moving the object further successively to several second locations of the plurality of second locations along further given paths. This allows positioning the object at several locations on the surface, wherein the actual position of the object is known, because the position of the first location and the given paths between the first location and a first second location and between the first second location and succeeding second locations are known.

The path can, for example, be given by defining a first and one or more second locations on the surface. This definition can, for instance, be performed on a model or an image of the surface, which can, for example, be displayed on a display unit. The model or the image of the surface can be registered with an actual image of the surface, which is, for example an image of an X-ray fluoroscopy system. The distance, in particular, the shortest distance, between the different first and second locations on the registered model or image define preferentially the paths for a movement from a first location to a first second location and preferentially from the first second location to the second location and so force.

It is preferred that the positioning device comprises a monitoring device for monitoring the position of the object on the surface. This allows monitoring, whether a desired location has been reached by the object. The monitoring device is, for example, an X-ray fluoroscopy system, which shows the position of the object on the surface on a projection image. The monitoring device can also be any other imaging device, which allows imaging the object on the surface.

It is further preferred that the positioning device is adapted for positioning the object being a first object on an inner surface of a second object. This allows positioning the object on the surface, even if the surface is difficult to reach, because the surface is a surface inside of a second object. For example, the positioning device can comprise a catheter, or the positioning device can be a part of a catheter system, in order to allow positioning the object being a first object on a surface within a second object, which is, for example, a technical object or a heart of a patient.

It is further preferred that the moving unit comprises a holding element for holding the object at a fixed position on the surface and motion elements for lifting the holding element from the surface, for moving the holding element and the object from the first position along the given path, and for lowering the holding element down to the surface at the second position, wherein at least one motion element is adapted for contacting the surface as long as at least one of the holding element and the object does not contact the surface. This allows moving the object along the given path with high accuracy.

In a preferred embodiment, the positioning device comprises at least one of a forcing unit for forcing the object against the surface and an orientation unit for orienting the object on the surface.

The forcing unit improves the contact between the object and the surface and, therefore, further improves the positioning of the object on the surface, in particular the accuracy and reliability of the positioning of the object on the surface. Furthermore, the orientation unit orientates, preferentially rotates, the object and preferentially also the moving unit before moving the object on the surface to a direction, in which the given path starts, wherein the movement of the object along an arbitrary given path is simplified.

It is further preferred that the positioning device comprises at least one of a force sensing unit for sensing the force between the object and the surface and an orientation sensing unit for sensing the orientation of the object with respect to the surface.

The force sensing unit allows monitoring the force between the object and the surface and, thus, monitoring the quality of the positioning of the object on the surface. Furthermore, the orientation sensing unit allows monitoring the orientation of the object with respect to the surface and, thus, monitoring the quality of the positioning of the object on the surface. If the force between the object and the surface or the orientation of the object with respect to the surface deviate from a corresponding desired force or orientation, respectively, the force between the object and the surface or the orientation of the object with respect to the surface can be corrected, for example, by using the forcing unit or the orientation unit, respectively.

In a preferred embodiment, the positioning device comprises an attaching unit for attaching at least one of the object and the positioning device to a frame. The frame is, for example, a part of a second object, which comprises a surface being the surface, on which the object being a first object has to be positioned.

The frame is, for example, a part of a second object, which comprises a surface, on which the object being a first object has to be positioned. If the object is a catheter tip, which has to be positioned on an inner wall of a heart of a patient, the catheter entry point to the arterial can be used as a fixed frame. Furthermore, in this case, the origin of the catheter outside the patient can be used as the frame, which is a fixed force frame. The attachment to the frame provides a stiffness so that the object can be pressed against the surface and oriented with respect to the surface with an improved control.

In a further aspect of the present invention a catheter apparatus is provided, wherein the catheter apparatus comprises a positioning device for positioning a first object being a tip of a catheter on an inner surface of a second object as claimed in claim 1.

In a further aspect of the present invention a positioning method for positioning an object on a surface is provided, wherein the positioning method comprises following steps:

providing the position of a first location on the surface,

moving the object on the surface from the first location to a second location along a given path, wherein the object is continuously in contact with the surface during the movement of the object on the surface from the first location to the second location.

In a further aspect of the present invention a catheter method is provided, wherein a first object being a tip of a catheter on an inner surface of a second object is positioned using the steps defined in claim 9.

In a further aspect of the present invention a computer program for positioning an object on a surface is presented, wherein the computer program comprises program code means for causing a computer to carry out the steps of the method as claimed in claim 6 when the computer program is carried out on a computer controlling a device as claimed in claim 1.

In a further aspect of the present invention a computer program is provided, wherein the computer program comprises program code means for causing a computer to carry out the steps of the method as claimed in claim 7 when the computer program is carried out on a computer controlling an apparatus as claimed in claim 5.

It shall be understood that the positioning device of claim 1, the catheter apparatus of claim 5, the positioning method of claim 6, the catheter method of claim 7, the computer program of claim 8 and the computer program of claim 9 have similar and/or identical preferred embodiments as defined in the dependent claims.

It shall be understood that preferred embodiments of the invention can also be any combination of the dependent claims with the respective independent claims.

### BRIEF DESCRIPTION OF THE DRAWINGS

These and other aspects of the invention will be apparent from and elucidated with reference to the embodiments described hereinafter. In the following drawings

FIG. 1 shows schematically a representation of a positioning device and a catheter apparatus in accordance with the invention,

FIG. 2 shows schematically a representation of a moving unit of the positioning device,

FIG. 3 shows schematically a first condition of the moving unit,

FIG. 4 shows schematically a second condition of a moving unit,

FIG. 5 shows schematically several parts of the positioning device and

FIG. 6 shows schematically a flowchart illustrating a positioning method for positioning an object on a surface in accordance with the invention.

### DETAILED DESCRIPTION OF EMBODIMENTS

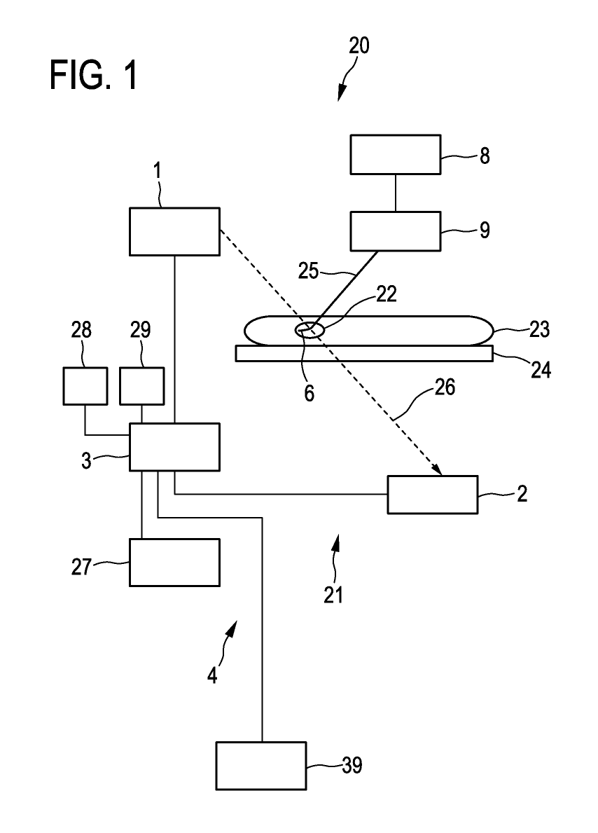

FIG. 1 shows schematically a representation of a catheter apparatus 20 comprising a positioning device 21 for positioning a first object 6 being a tip of a catheter on an inner moving surface of a second object 22 being in this embodiment a heart of a patient 23 located on a patient table 24. The catheter apparatus 20 comprises a catheter 25, which is controlled by a control unit 9. The catheter 25 and the catheter tip 6 can comprise ablation and/or sensing electrodes for ablating tissue of the heart and for sensing the tissue. The positioning device 21 comprises a provision unit 4 for providing the position of a first location on a moving surface of the second object 22. The provision unit 4 is, in this embodiment, an X-ray fluoroscopy system comprising an X-ray source 1, an X-ray detection unit 2 and a control unit 3 for controlling the X-ray fluoroscopy system, in particular for controlling the X-ray source 1 and the X-ray detection unit 2. The radiation 26 of the X-ray source 2 passes the second object 22 including the first object 6 on a moving surface of the second object 22 at a first location, and the radiation 26 after having passed the second object 22 and the first object 6 is detected by the X-ray detection unit 2. The control unit 3 generates a projection image from the detected X-ray radiation 26 and displays the projection image on a display unit 27. On the display unit 27 the first location of the first object 6, which is in this embodiment the catheter tip 6, on the moving surface can directly by seen. Furthermore, the provision unit 4 preferentially comprises a first location determination unit 29, which is adapted for determining the first location from the projection image. Since the catheter tip 6 can clearly be identified on the projection image, the actual position of the catheter tip, which is the first location, can be determined by, for example, thresholding.

Furthermore, the provision unit 4 comprises an input unit 28, which is adapted for allowing a user to input a first location by using, for example, a graphical user interface into the control unit 3 and on the projection image. The input unit 28 comprises preferentially a mouse and/or a keyboard. The input unit 28 and the control unit 3 can also be adapted for allowing a user correcting the determined first location on the display unit 27 using, for example, a graphical user interface.

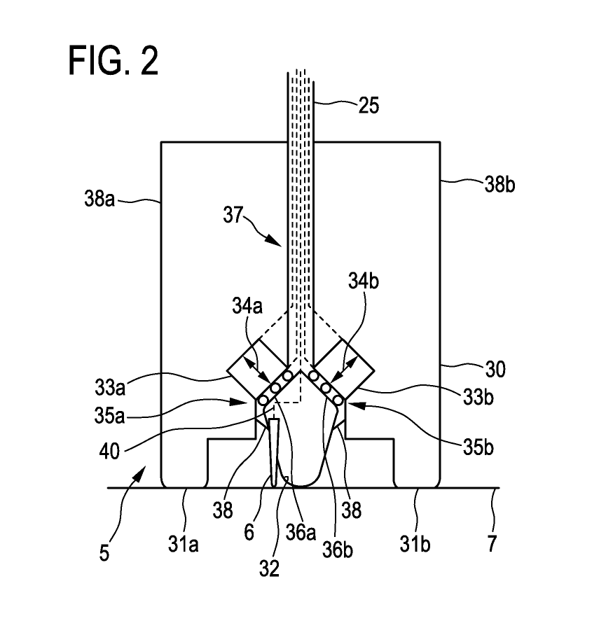

The positioning device 21 further comprises a moving unit 5, which is located adjacent to the first object 6 and which, in this embodiment, is located at the distal portion of the catheter 25. An embodiment of a moving unit 5 is schematically shown in more detail in FIG. 2.

The moving unit 5 comprises a moving body 30 having two parts or fingers 38a, 38b with protrusions 31a, 31b for contacting the moving surface 7. The moving unit 5 further comprises a holding element 32, which is movable with respect to the moving body 30. For moving the holding element 32 with respect to the moving body 30 the moving unit 5 further comprises at least two piezo crystals 33a, 33b. The piezo crystal 33a is designed such that it extends and contracts in the direction 34a, and the piezo crystal 33b is designed such that it extends and contracts in the direction 34b. The piezo crystals 33a, 33b are coupled with the holding element 32 via bearings 35a, 35b, which are preferentially sliding bearings or rolling bearings. The bearings 35a, 35b are located on oblique surfaces 36a, 36b of the holding element 32, which are inclined with respect to the normal direction of the moving surface 7, wherein the oblique surface 36a is inclined in one direction and wherein the oblique surface 36b is inclined in the opposite direction with respect to the normal direction of the moving surface 7. The piezo crystals 33a, 33b are rigidly attached to the moving body 30. Furthermore, the piezo crystals 33a, 33b are contacted via wires 37 with a controllable voltage source (not shown in FIG. 2) for applying an electrical field to the piezo crystals 33a, 33b such that the piezo crystals 33a, 33b can be extended or contracted in the directions 34a, 34b, respectively. The moving unit 5 further comprises a forcing element 38, which is also schematically shown in FIG. 2 and which is, for example, a spring. The forcing element 38 is connected to the moving body 30 and the holding element 32 such that the forcing element 38 urges the holding element 32 towards the moving body 30, i.e. in FIG. 2 the holding element 32 is urged upwards by the forcing elements 38. In this embodiment, the moving unit 5 comprises two forcing elements 38. But in other embodiments, the moving unit can also comprises one or more than two forcing elements.

The first object 6, i.e. in this embodiment the catheter tip 6, is attached to the holding element 32. In other embodiments, the first object 6 can also be attached to another part of the moving unit 5.

The catheter tip 6 is connected to the control unit 9 via a wire 40. The first object 6 can also be connected to the control unit 9 via several wires.

The holding element 32 can be formed as a chuck. The chuck ensures that the moving unit 5 and, therefore, the first object 6, will not move with respect to the moving surface 7, for example, due to friction force and/or suction force. The moving body 30 is divided into two parts 38a, 38b.

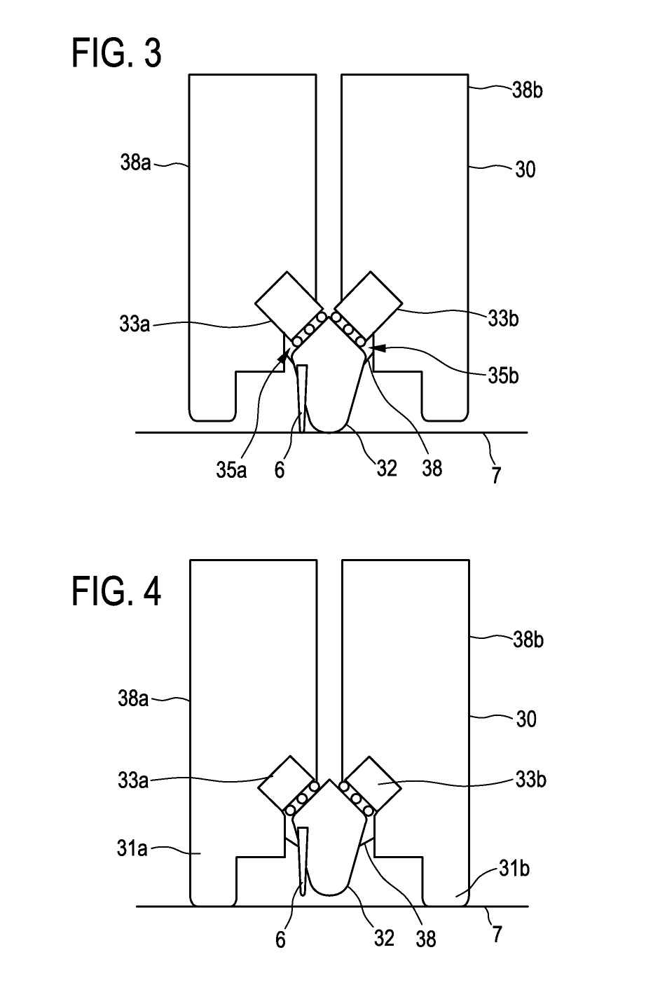

At a first location on the moving surface 7 the moving unit is preferentially in a condition schematically shown in FIG. 3. In FIG. 3, the holding element 32 is fixed to the moving surface 7 and the parts 38a, 38b of the moving body 30a are lifted from the moving surface 7. In order to move the first object 6 along a given path from the first location to a second location, the piezo crystals 33a, 33b are controlled such that the moving body 30, i.e. the parts 38a, 38b of the moving body 30, are moved towards the moving surface until the protrusions 31a, 31b contact the moving surface 7 and the condition shown in FIG. 2 is reached. Then, the piezo crystals 33a, 33b are controlled such that the holding element 32 is lifted from the moving surface 7, and the condition shown in FIG. 4 is reached. The piezo crystals 33a, 33b are then controlled such that the holding element 32 having attached the first object 6 is moved along the given path from the first location to a second location, i.e. the holding element 32 and the first object 6 are moved with respect to the moving surface 7 and with respect to the two parts 38a, 38b of the moving body 30, while the protrusions 31a, 31b are still in contact with the moving surface 7, in order to keep the spatial relationship between the moving surface 7 and the moving unit 5 and, therefore, between the moving surface 7 and the object 6. The protrusions 31a, 31b are preferentially also formed as chucks. At the second location the holding element 32 and, therefore, the first object 6, are moved towards the moving surface 7, until the holding elements 32 and the first object 6 contact the moving surface 7. Then, the piezo crystals 33a, 33b are controlled such that the parts 38a, 38b are lifted from the moving surface 7 and also moved to the second location, until the condition shown in FIG. 2 is reached.

For a purely up-down-movement, preferentially the piezo crystals 33a, 33b contract or extend simultaneously in the same way, respectively. For a movement having a component parallel to the moving surface 7, preferentially the piezo crystals 33a, 33b contract or extend differently, respectively.

The parts or fingers 38a, 38b, the forcing elements 38 and the piezo crystals 33a, 33b are motion elements for lifting the holding element from the moving surface, for moving the holding element and the object from the first position along the given path and for lowering the holding element down to the moving surface at the second position.

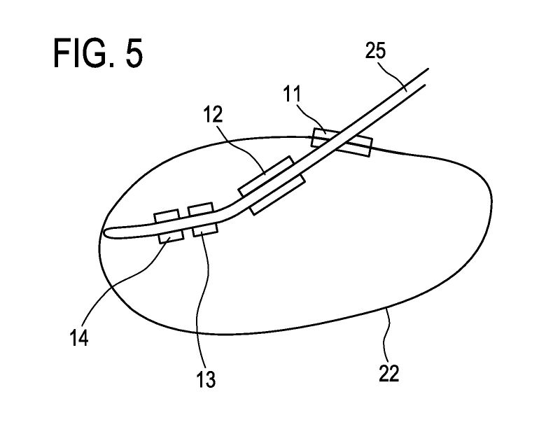

FIG. 5 shows schematically further preferred components of the positioning device 21. A positioning device 21 preferentially further comprises an attaching unit 11 for attaching the positioning device, in this embodiment via the catheter 25, to a frame. The frame is in this embodiment the catheter entry point to the arterial. The attaching unit 11 is, for example, a clamp for clamping the catheter 25 to the frame. The positioning device preferentially further comprises a forcing unit 12, which comprises, for example, one or several springs, for urging the moving unit 5 and the first object 6 against the moving surface 7. Furthermore, the positioning device 21 comprises preferentially an orientation unit 13, which orientates, in particular, rotates the moving unit 5, and thus the first object 6, in order to simplify movements of the moving unit 5 and the first object 6 along arbitrary given paths. The positioning device 21 further preferentially comprises an extendable and contractable element 14, which is, for example, constructed as a telescopic element, in order to adapt the length of the catheter 25 to the distance between the attaching unit 11 and the desired location on the moving surface.

Preferentially, the positioning device 21 further comprises a force sensing unit and an orientation sensing unit for sensing the force between the first object 6 and the moving surface 7 and the orientation of the first object 6 with respect to the moving surface.

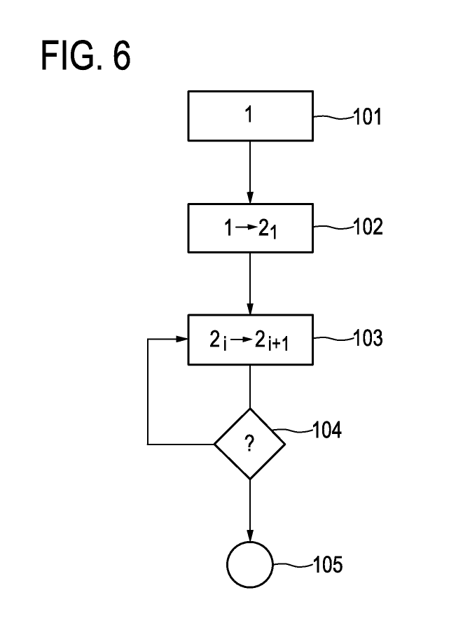

An embodiment of the positioning method for positioning an object on a surface will now be described in more detail with reference to a flowchart shown in FIG. 6. In step 101, the moving unit 5 and the first object 6 are located on the moving surface 7 at a first location, which is provided by the provision unit 4, which is, in this embodiment, an X-ray fluoroscopy system.

In step 102, the moving unit 5 comprising the first object 6 is moved, as described above, from the first location along a given path to a second location. At this second location, and also at the first location and at further second locations, the first object 6, which is in this embodiment a catheter tip, can, for example, ablate and/or sense tissue of the heart. In step 103, the moving unit 5 and, therefore, the first object 6 is further moved from the second location being a first second location to a further second location along a given path. Also at the second location the catheter tip 6 can ablate and/or sense tissue of the heart. In step 104 it can be decided, whether the movement should proceed to further second locations or whether the movement should stop. This decision can be inputted via an input unit like a keyboard or a mouse in the positioning device 21 or the positioning device 21 can stop the movement, after a predetermined stop criterion has been reached. This criterion is, for example, that the moving unit 5 and, therefore, the first object 6 have moved to all of a given set of first and second locations. The positioning method ends in step 105.

Although, in the above described embodiment, only two parts 38a, 38b of the moving body 30 or two moving fingers are provided, in accordance with the invention the positioning device can also comprise more than two parts or two fingers.

Since during the movement the first object remains via the moving unit 5 indirectly in contact with the moving surface 7 and since the step size is known, for example, by design or experience, the first object 6 can be moved confidentially over the moving surface without continuously measuring its position, for example, by the X-ray fluoroscopy system 4. Line segments, in particular scars, can be made by moving the first object 6, which is, in the above described embodiment, a catheter tip 6, over the surface while firing the ablation tip.

The provision unit 4 preferentially further comprises a registration unit 39 for registering the position of the first object 6 at the first location with the projection image of the X-ray fluoroscopy system. Furthermore, the provision unit can be operated as a monitoring unit 4, in order to monitor at intermediate second locations, whether the actual position of the first object 6 still coincides with a position of the first object 6 determined by the position determination unit 8, which determines the actual position of the first object 6 preferentially by vectorially adding the provided first location and the one or several consecutive given paths. As a monitoring unit also a separate monitoring unit can be used, which is separated from the provision unit. The monitoring unit and/or the provision unit can also be an ultrasound imaging device, another X-ray imaging device or a magnetic resonance imaging device, which images the first object 6 on the moving surface 7 for determining the position of the first object 6 on the moving surface 7.

Cornering is preferentially performed by rotating the catheter, i.e. by changing the start forward direction of the movement of the moving unit, wherein preferentially separate parts of the moving unit or separate fingers are controlled independently.

The orientation unit and/or the forcing unit use preferentially a flexible joint and/or a catheter segment that provides orientation to the tip and a normal force for tip-tissue contact, without disturbing the planar motion of the tip. This joint might be a passive or actuated one and it preferentially follows the tip. The flexible joint can also be realized by the flexibility of a catheter material.

Preferentially the holding element 32 and/or the first object 6 have a curved surface to become less sensitive for orientation errors with respect to the moving surface 7.

Preferentially the parts 38a, 38b of the moving body 30 or the fingers are controlled such that, if the movement is disturbed and if this is detected, for example, by the orientation sensing unit and/or the force sensing unit, the movement of these parts of fingers is corrected in order to move the moving unit and the first object along the given path.

A feedback for moving the first object, i.e. a provision unit for providing the provision of the first location on the moving surface and/or a monitoring unit, can be provided, for example, by an ultrasound imaging device, which is, for example, located in an arterial, in particular in another arterial, in which the catheter is not present. This can be done by a linear phased array on a second catheter. Preferentially, the linear phased array is rotated in order to form an image that can be used for determining the position of the catheter tip being the first object 6, which has to be moved. Furthermore, a displacement sensor in the first object can be used for providing feedback for moving the tip.

If the first object is a catheter tip comprising sensing and ablation functionality, the positioning device can be adapted such that the stepped path is stored and that the catheter tip moves along the given path firstly forward in one direction and secondly backwards along the opposite direction along the given path, wherein ablation can be performed during the forward movement and sensing can be performed during the backward movement.

In another embodiment of the positioning device, one or more wires through the catheter can be used, in order to convey translational and rotational forces and movements to the first object. These movements are converted locally to the wanted motion of the moving unit, in particular the wanted motion of the two parts of the moving unit or the fingers. This is preferentially implemented by using a disc. This disc has finger elements mounted to its surface off-axis. This disc can rotate and translate driven by the wires. The direction of the planar movement of the tip can be selected by rotating the wire left or right.

In another embodiment, the parts of the moving body or the fingers can be moved using motors, for example, a translational and a rotational motor, to obtain the wanted motion of the fingers, while the first object, which is for example a catheter tip, remains directly or indirectly via the moving unit in contact with the moving surface.

In another embodiment, magnets are used for providing a normal force, which holds the first object on the moving surface. For example, the first object can be a magnetisable object or can comprise a magnetisable material or can be or can comprise a magnet, in particular a permanent magnet, and on the opposite side of the moving surface, for example, outside of a patient, a corresponding magnet can be used for providing a normal force, which holds the first object on the moving surface.

It is preferred, that the moving unit is adapted such that the distance between a first location and a second location and between the second location and a consecutive second location is in the range of 0.5 to 1.5 mm, further preferred in the range of 0.8 to 1.2 mm, and it is further preferred that the moving unit is adapted such that this distance is 1 mm.

Preferentially, the holding element 32 is a chuck, which is statically attached to the first object, which is preferentially a catheter tip.

Although in the above described embodiment the catheter tip has been described as a catheter tip providing electrical energy for ablation purposes, the first object can also be another kind of catheter tip. For example, the catheter tip can provide light energy of a laser for ablation purposes. The moving unit can be realized by using regular catheter tip materials, for example, barium polyurethane.

Although in the above mentioned embodiments the object has been positioned on a moving surface, the positioning device for positioning an object on a surface can also position an object on a surface, which is not moving. The positioning device can be used for positioning an object on any surface. In particular, the positioning device can position an object on a surface accurately, even if the object and/or the surface has to withstand the flow of fluids like blood or other influences acting on the object and/or the surface. Other variations to the disclosed embodiments can be understood and effective by those skilled in the art and practicing the claimed invention, from a study of the drawing, the disclosure and the dependent claims. In the claims, the word “comprising” does not exclude other elements or steps, and the indifferent article “a” or “an” does not exclude a plurality.

A single unit may fulfill the functions of several items recited in the claims. The mere effect that certain measures are recited in mutually different dependent claims does not indicate that a combination of these measures can not be used to advantage.

A computer program may be stored/distributed on a suitable medium, such as an optical storage medium or a solid-state medium, supplied together with or as part of other hardware, but may also be distributed in other forms, such as via the internet or other wired or wireless telecommunication systems.

Any reference signs in the claims should not be construed as limiting the scope.

1. A positioning device for positioning an object on a surface, the positioning device comprising:
a provision unit (4) for providing the position of a first location on the surface,
a moving unit (5) for moving the object (6) on the surface (7) from the first location to a second location along a given path, wherein the object (6) is continuously in contact with the surface (7) during the movement of the object (6) on the surface (7) from the first location to the second location.

2. The positioning device as claimed in claim 1,
wherein the positioning device further comprises a position determination unit (8) for determining the position of the second location on the surface (7) from the provided position of the first location and from the given path.

3. The positioning device as claimed in claim 1,
wherein the moving unit (5) is adapted for moving the object (6) from the first location to a second location of a plurality of second locations along a first given path and for moving the object (6) further successively to several second locations of the plurality of second locations along further given paths.

4. The positioning device as claimed in claim 1,
wherein the moving unit (5) comprises
a holding element (32) for holding the object (6) at a fixed position on the surface (7) and
motion elements (38a, 38b, 38, 33a, 33b)
for lifting the holding element (32) from the surface,
for moving the holding element (32) and the object (6) from the first position along the given path, and
for lowering the holding element (32) down to the surface (7) at the second position, wherein at least one motion element is adapted for contacting the surface (7) as long as at least one of the holding element (32) and the object (6) does not contact the surface (7).

5. A catheter apparatus comprising a positioning device (21) for positioning a first object (6) being a tip of a catheter on an inner surface (7) of a second object (22) as claimed in claim 1.

6. A positioning method for positioning an object (6) on a surface (7), the positioning method comprising following steps:
providing the position of a first location on the surface (7),
moving the object (6) on the surface (7) from the first location to a second location along a given path, wherein the object (6) is continuously in contact with the surface (7) during the movement of the object (6) on the surface (7) from the first location to the second location.

7. A catheter method,
wherein a first object (6) being a tip of a catheter on an inner surface (7) of a second object (22) is positioned using the steps defined in claim 6.

8. A storage device for a computer used for positioning an object (6) on a surface (7), the storage device comprising program code means for causing the computer to provide the position of a first location on the surface (7), and determine movement of the object (6) on the surface (7) from the first location to a second location along a given path, wherein the object (6) is continuously in contact with the surface (7) during the movement of the object (6) on the surface (7) from the first location to the second location being configured to control a positioning device for positioning an object on the surface, the positioning device including a provision unit (4) for providing the position of the first location on the surface, and a moving unit (5) for moving the object (6) on the surface (7) from the first location to the second location along the given path, the object (6) being continuously in contact with the surface (7) during the movement of the object (6) on the surface (7) from the first location to the second location.

9. A storage device for a computer, the storage device comprising program code means for causing the computer to position a tip (6) of a catheter on an inner surface (7) of a second object (22), the positioning including providing a position at a first location on the surface (7) and moving the tip (6) on the surface (7) from the first location to a second location along a given path, on which path the tip (6) is continuously in contact with the surface (7) during the movement of the tip (6) on the surface (7) from the first location to the second location, the computer being configured to control a positioning device for positioning the tip (6) on the surface (7), the positioning device including a provision unit (4) for providing the position of the first location on the surface, and a moving unit (5) for moving the tip (6) on the surface (7) along the given path from the first location to the second location, the tip (6) being continuously in contact with the surface (7) during the movement of the tip (6) on the surface (7) from the first location to the second location.
